(19) United States
(12) Patent Application Publication
(10) Pub. No.: US 2024/0415829 A1
(43) Pub. Date: Dec. 19, 2024

(54) METHODS AND COMPOSITIONS FOR THE TREATMENT OF DRUG OVERDOSE

(71) Applicant: Brown University, Providence, RI (US)

(72) Inventors: Elinore McCANCE-KATZ, Cranston, RI (US); Carolina L. HAASS-KOFFLER, Providence, RI (US)

(21) Appl. No.: 18/742,945

(22) Filed: Jun. 13, 2024

Related U.S. Application Data
(60) Provisional application No. 63/508,236, filed on Jun. 14, 2023.

(51) Int. Cl. A61K 31/475; A61K 31/485; A61P 39/02

(52) U.S. Cl. CPC A61K 31/475; A61K 31/485; A61P 39/02

(57) ABSTRACT

Described herein are lifesaving compositions and methods including the administration of alpha-2 (α₂) adrenergic antagonists as a drug class for use in the reversal of substance overdose that can be contributed to by xylazine and xylazine congeners, alcohol, fentanyl/opioids, and benzodiazepines in humans. In some instances, the substance overdoses can appear to be opioid induced but are not responsive to administration of therapeutic agents (e.g., Naloxone administration). The antagonists can be safely and quickly produced using yohimbine, and the compositions and methods are operative to address the United States' national crisis of human overdoses caused by combining various illicit (street) drugs with xylazine.

### CROSS-REFERENCE TO RELATED APPLICATIONS

This application claims benefit and priority from U.S. Provisional Patent Application No. 63/508,236, filed on 14 Jun. 2023, which is incorporated by reference herein in its entirety.

### FIELD OF THE INVENTION

The embodiments of the present invention relate to novel approaches to preventing and/or reversing serious and otherwise lethal drug overdoses, which in some instances can appear to be opioid induced but are not responsive to administration of therapeutic agents (e.g., Naloxone administration).

### BACKGROUND OF THE INVENTION

The Biden-Harris Administration via the White House of the United States, recently (April 2023) issued a warning that xylazine contamination of illegal street fentanyl in the United States is an emerging national threat.¹ Xylazine is a non-opioid tranquilizer approved by the United States (US) Food and Drug Administration (FDA) for veterinary use (e.g., in horses, cattle, sheep) but not for human use. Xylazine is an agonist of the alpha-2 (α₂) adrenergic receptor.

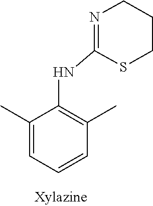

Xylazine is most frequently found in combination with fentanyl. The U.S. Drug Enforcement Administration (DEA) reported in 2022 that 23% of fentanyl powder and 7% of fentanyl pills seized by law enforcement contained this drug.² Xylazine is also frequently found in drug combinations known by the street name “speedball”, which is a mixture of several drugs—usually cocaine, with either heroin, morphine, or fentanyl, wherein xylazine can quickly cause drastic skin ulcerations and lesions in humans, along with other health issues.³ Even used on its own, xylazine is a deadly drug in the United States, often called the “flesh eating drug”, where it is also known by the street name “tranq” or “anestesia de caballo” (The Spanish Language for “anesthetic of horse”), particularly in Puerto Rico.⁴ Tragically, there are currently no xylazine antidotes in use for human use.^{5,6} As a result of this and other reasons (e.g., lack of testing, lack of public awareness), the number of drug overdose deaths in the United States has been steadily rising since 2019. Between 2020 and 2021, forensic laboratory identifications of xylazine rose in all four US census regions, most notably in the Census South Region (193%) and the West Region (112%). The DEA has reported that between 2020 and 2021, xylazine-positive overdose deaths increased by 1,127% in the Census South Region, 750% in the West Region, more than 500% in the Midwest Region, and more than 100% in the Northeast Region.¹ This increase in overdose deaths is based on forensic diagnosis because there are currently no easily accessible lab tests available for detecting xylazine, and the DEA has established that most xylazine in the US street drug supply is not ever identified at the time a drug seizure is made.⁵ In these cases, xylazine is only detected through subsequent testing conducted by forensic laboratories after the overdose death. As such, the adverse events caused by xylazine use and its prevalence across the United States are very likely grossly underreported.⁵ This situation is a major public health emergency.

Accordingly, there is an urgent need for new methods for emergency treating of subjects, for saving lives, and for preventing overdose in situations of suspected or known drug overdose.

### BRIEF SUMMARY OF THE INVENTION

Example embodiments of the present invention provide improved technology (e.g., health care systems, deployments, and strategies) for use in preventing and/or in treating suspected or established drug overdose emergencies. For example, the invention provides the use of α₂ adrenergic antagonists as a drug class for use in the reversal of drug overdose contributed to by xylazine, xylazine congeners, alcohol, and/or benzodiazepines in humans. In some embodiments, the invention discloses administration of one or more α₂ adrenergic antagonists in addition to or in place of a different treatment. In some embodiments, the invention provides administration of yohimbine in addition to, or in place of, a different treatment. An example of the different treatment can consist of only administration of Naloxone and supportive therapy. As a medication, yohimbine has already been approved by the FDA to treat impotence in humans. The chemical structure of yohimbine is shown below.

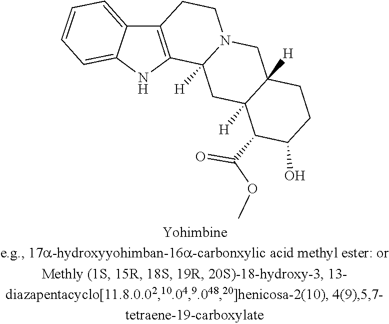 [SMILES: COC(=O)[C@@H]1[C@H]2C[C@H]3c4[nH]c5ccccc5c4CCN3C[C@@H]2CC[C@@H]1O]

The doses of yohimbine to be used to prevent or treat drug overdose have been used in research studies, thereby providing safety data indicating that the yohimbine (or a salt thereof) may be safely given in an overdose situation. As an example, yohimbine is a generic drug available over-the-counter (OTC), and cost can be relatively low. It is administered without issues and can be taken prophylactically. It can also be combined with Naloxone to make a product that will better address the contributing factors to drug overdose.

The present invention, in one of its embodiments, provides a method for treating a subject that is experiencing a substance overdose (e.g., heroin, fentanyl, or other opioids), or is suspected of experiencing a drug overdose. According to some aspects, the one or more drug(s) in the substance are unknown.

The method comprises the step of administering to the subject an α₂ adrenergic antagonist, whereby one or more signs or symptoms of the drug overdose are modulated, changed, and/or reversed. The term “suspected” can include previously diagnosis of the overdose or previously diagnosed as having a likelihood of the overdose (e.g., a person with an addiction).

In some embodiments, the α₂ adrenergic antagonist is an antagonist of a receptor of α₂ adrenergic, or a precursor of the antagonist. In alternative embodiments, the α₂ adrenergic antagonist is a blocker, an orthosteric antagonist, allosteric antagonist, conformation changing binder, a modulator, a partial inverse agonist/inverse agonist, or a combination thereof. In some embodiments, the α₂ adrenergic antagonist is yohimbine or a pharmaceutically acceptable salt thereof.

In some embodiments, the one or more signs or symptoms of substance overdose include, but are not limited to, dry mouth, drowsiness, hypertension and/or hypotension, tachycardia, bradycardia, hyperglycemia, reduced heart rate, hypothermia, coma, respiratory depression, and/or dysrhythmia.

In some embodiments, the administering of the α₂ adrenergic antagonist causes a partial or complete reversal of the symptoms of the substance overdose at least contributed to by one or more of, included in said substance, xylazine and xylazine congeners, alcohol, fentanyl/opioids, and benzodiazepines. According to some aspects, (e.g., the emerging national emergency), the substance being abused can further comprise fentanyl and/or other opioid(s). As such, in some embodiments, the α₂ adrenergic antagonist is co-administered with a more conventional substance overdose treatment, e.g., Naloxone (Brands: NARCAN® and EVZIO®) for narcotic overdose, via the same route of administration, via different routes of administration, or in the same therapeutic formulation. In alternative embodiments, the α₂ adrenergic antagonist is before or after the more conventional substance overdose treatment. For example, the subject can be nonresponsive to an emergency Naloxone administration even though an opioid overdose is suspected. In this example, the α₂ adrenergic antagonist subsequently administered, and one or more signs or symptoms improve. Unbeknownst to health care providers, the subject was exposed to xylazine, which was only apparent after execution of the methods disclosed herein. Because alpha 2 adrenergic antagonists are general stimulants for cardiovascular and respiratory function, they may be helpful in any unknown drug overdose including opioids (this does not mean to imply that it is more effective than naltrexone, only that administration of an alpha 2 adrenergic antagonist is likely to be helpful in an overdose situation).

According to some aspects, the α₂ adrenergic antagonist administered is a fast-acting formulation capable of elevating α₂ adrenergic antagonist plasma concentration levels about ≥50% of peak concentrations before about 60 minutes, optionally before about 30 minutes, or before about 15 minutes, or before about 10 minutes, or before about 5 minutes, or before about 4 minutes, or before about 3 minutes, after having been administered to the subject.

In some embodiments, the formulation is provided to the subject in a form of an oral tablet, buccal film, sublingual tablet, nasal spray, inhaled aerosol, intravenous injectable, intra-arterial injectable, intracardiac injectable, hydrogel or organogel, powder, intramuscular injectable, subcutaneous injectable, intradermal injectable, eye (ocular) formulation drops, ocular mist, or fast absorbing topical formulation. According to some aspects, the formulation can be administered by a non-health care provider in an emergency or in a non-healthcare (field administration) setting.

Accordingly, in some embodiments, the method for treating a substance overdose first comprises the sole administration of Naloxone to the subject. If this Naloxone treatment is ineffective in reversing or reducing the signs or symptoms of the drug overdose, subject is further administered an α₂ adrenergic antagonist. In alternative embodiments, the Naloxone treatment is co-administered with the α₂ adrenergic antagonist, either separately or combined in the same formulation.

According to some aspects, the methods include administering the α₂ adrenergic antagonist prophylactically to a subject at risk of a substance overdose, wherein the substance comprises one or more of xylazine and xylazine congeners, alcohol, fentanyl/opioids, and benzodiazepines. In some embodiments, the subject at risk of a substance overdose has a history of substance abuse and/or exhibits one or more signs or symptoms of soft tissue injuries, skin ulcers, abscesses, and/or tissue necrosis. The α₂ adrenergic antagonist is administered prophylactically in a pharmaceutically effective amount to prevent symptoms of substance overdose upon exposure to substance comprising one or more of xylazine and xylazine congeners, alcohol, fentanyl/opioids, and benzodiazepines.

In some embodiments, the present invention provides a product for fast-acting use in humans comprising GMP (Good Manufacturing Practice as enforced by the FDA) quality yohimbine or a pharmaceutically acceptable salt thereof, wherein said product is capable of providing a field administration including the administration outside of a healthcare facility. According to some aspects, the product further comprises Naloxone.

In some embodiments, the product disclosed herein is fast-acting, capable of providing yohimbine (or other antagonist) plasma concentration levels≥50% of peak concentrations before about 60 minutes after an administration of the product to a human subject, before about 30 minutes after an administration, before about 10 minutes, before about 5 minutes, before about 4 minutes, or before about 3 minutes. According to some aspects, said product includes a formulation in a form of an oral tablet, buccal film, sublingual tablet, nasal spray, inhaled aerosol, intravenous injectable, intra-arterial injectable, intracardiac injectable, hydrogel or organogel, powder, intramuscular injectable, subcutaneous injectable, intradermal injectable, eye (ocular) formulation drops, ocular mist, or fast absorbing topical formulation (e.g., with dimethyl sulfoxide quick skin penetrant or nanoparticles/gels operative to traverse intercellular spaces).

The product can be configured, according to some aspects, for emergency use in humans. In another example, the product is provided over the counter, with a shelf life (at room temperature or 25° C.) of greater than 6 months, greater than a year, greater than two years, or greater than five years. The product can be provided in a formulation with preservatives (e.g., the parabens). The product can be sterile filtered and sealed and provided without preservative. The product can be sealed and irradiated.

In some embodiments, the product disclosed above is wherein the product consists of yohimbine (or the salt thereof) or consists essentially of yohimbine (or the salt thereof) such that only other ingredients are excipients and not an additional API (active pharmaceutical ingredient). According to some aspects, the product consists essentially of yohimbine and Naloxone, such that only other ingredients are excipients and not an additional API.

Depending on the formulation, delivery, mode of administration, and packaging, the product can be about 0.1 mg/mL to about 100 mg/mL yohimbine (or a salt thereof), about 1 mg/mL to about 50 mg/mL yohimbine, about 5 mg/mL to about 40 mg/mL yohimbine, about 10 mg/mL to about 30 mg/mL yohimbine, or about 20 mg/mL yohimbine configured for intravenous or intramuscular administration with sterile syringes for field administration. It is to be understood that the pharmaceutical salts described herein can be used any time the antagonists of the present technology are discussed.

In some embodiments, the present invention provided a combination product including Naloxone and yohimbine operative to be administered via intravenous, intramuscular, or intranasal route in preventing and/or treating drug overdose where the drug(s) ingested are unknown. According to some aspects, the Naloxone and yohimbine are provided in a liquid form.

In some embodiments, a deployable product is disclosed including a formulation of an α₂ adrenergic antagonist and/or an antagonist of a receptor of α₂ adrenergic, or a precursor of said antagonist, said formulation provided in a form of an oral tablet, buccal film, sublingual tablet, nasal spray, inhaled aerosol, intravenous injectable, intra-arterial injectable, intracardiac injectable, hydrogel or organogel, powder, intramuscular injectable, subcutaneous injectable, intradermal injectable, eye (ocular) formulation drops, ocular mist, or fast absorbing topical formulation. According to some aspects, the deployable product is wherein the formulation includes yohimbine and optionally Naloxone.

In some embodiments, a deployable kit is disclosed herein including any of the products disclosed above and instructions for use. The instructions can include any step(s) of the methods disclosed herein.

In some embodiments, the invention provides a system for preventing substance overdose, likelihood of substance overdose, and/or one or more deaths by substance overdose in a population of humans, said system comprising providing any steps of the method(s) disclosed herein, the yohimbine, the formulation, the deployable product(s), and/or the kit(s), to at least one member of the population.

In some embodiments, a method of diagnosing a human subject for an exposure to xylazine is provided herein, said method comprising the steps of: (1) administering an α₂ adrenergic antagonist and/or an antagonist of a receptor of α₂ adrenergic, or a precursor of said antagonist to the subject; and (2) monitoring one or more signs or symptoms for a modulation, change, or reversal in the subject; whereby the presence of the modulation, change, or reversal of symptoms indicates the exposure to xylazine. According to some aspects, the method is wherein said antagonist comprises or consists essentially of yohimbine and/or Naloxone. The method of diagnosing can be performed, according to some aspects, wherein before execution of step (1), the subject has previously had an administration of Naloxone and a monitoring of one or more signs or symptoms for a modulation/change/reversal in the subject; and there was not the presence of said modulation/change/reversal after the administration of the Naloxone. In some embodiments, the “exposure to xylazine” is an unidentified overdose.

In an alternative aspect, the present invention provides a long-term method of preventing a drug overdose in a subject in need thereof, by a long term administration of a therapeutically effective amount of an α₂ adrenergic antagonist (e.g., yohimbine).

The invention contemplates that the above-described compounds of yohimbine can be derivatized or can be structurally altered, for example, by addition or substitution of one or more atoms using a radioisotope or using a different element (e.g., B in place of C), by removal of an ester or by addition of a salt form, an amino acid, a sugar, or a peptide. Hydrates and/or solvates can be formed by 1) dissolving the antagonist or yohimbine in water and/or solvent and slowly drying, whereby water and/or solvent remain hydrogen bonded with OH groups in the molecule. In some embodiments, the above-described compounds are attached to or associated with a targeting moiety. In some embodiments, the targeting moiety is a particle or an antibody with affinity for a specific type of cell.

Other implementations are also described and recited herein.

### BRIEF DESCRIPTION OF THE DRAWINGS

Solely for the purpose of illustration, certain embodiments of the present invention are explained using examples in the drawings described below. It should be understood, however, that the invention is not limited to the precise arrangements, dimensions, and configurations shown. In the drawings:

FIG. 1 provides a photo of xylazine injectable ROMPUN® (Bayer Pharmaceuticals tradename) for horse sedative, FDA-approved for use in horses (strictly not for human use). At left is a syringe accessible bottle 6 with a septum at top. At right is an example packaging 8 showing ingredients of xylazine hydrochloride, methylparaben, propylparaben (preservatives), water, citric acid/sodium citrate for pH adjustments.

FIG. 2A-C provides an example illustration of an FDA-approved NARCAN© (Naloxone HCl) nasal spray 4 mg. FIG. 2A provides a photo of the nasal spray packaged in a box 10. FIG. 2B provides a photo of a nasal spray device 14 out of the box. FIG. 2C provides a photo of a hand discharge of the nasal spray from the device 18. The nasal spray (NARCAN®) has been approved for over the counter sale and distribution as of Mar. 29, 2023.⁷

FIG. 3 provides a photo of a kit for providing α₂ adrenergic antagonist and/or an antagonist of a receptor of α₂ adrenergic, or a precursor of said antagonist in an example kit container 30. The container 30 can contain a nasal spray as depicted in FIG. 2, and/or a syringe 32, needles 34, and ampule 36. The container 30 can contain a bottle 40 as the example in FIG. 4. The ampule 36 can be for repeated use as shown in the accessible bottle in FIG. 1.

FIG. 4 provides a photo of fast-acting nitroglycerin, Nitrostat® (Pfizer Pharmaceuticals tradename) for FDA-approved use in humans as an example of tablets 42, sublingual tablets 42, and/or capsules 44 in a bottle 40. Nitroglycerin is an example of the decades old fast-acting sublingual and provides an example of a fast-acting sublingual that can be achieved with the antagonists disclosed herein.

FIG. 5A-B provides an example of yohimbine for sale in the United States. FIG. 5 shows a photo of NUTRICOST® Yohimbine for sale at Amazon.com⁸ (accessed Jun. 6, 2023). FIG. 5A shows a bottle 51 containing capsules. FIG. 5B shows the capsules are formulated 50 with rice flour in capsule (gelatin).

FIG. 6A, FIG. 6B, FIG. 6C, FIG. 6D, and FIG. 6E each show data for effects of yohimbine (32.4 g) compared to placebo in the laboratory in patients with opioid use disorder.

All trademarks, images, likenesses, words, and depictions in the drawings are plainly in fair use and are provided solely for the purposes of illustration of the invention in view of national need further discussed in detail below.

### DETAILED DESCRIPTION OF THE INVENTION

The subject innovation is now described in some instances, when necessary, with reference to the drawings. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail. In general, chemical terminology is found in the International Union of Pure and Applied Chemistry GoldBook.⁹ This disclosure is purposefully in plain words, known to a person of skill in the art, but Merriam-Webster's Online Dictionary is used, when appropriate, for terms not specifically demonstrated herein or not known in the art.¹⁰

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation. The term “including” can be interchanged with “comprising”.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention. For example, a pharmaceutical formulation can consist essentially of yohimbine, meaning that a variety of excipients or other additives can be present in the formulation, but no other active pharmaceutical ingredient (API) is present in the formulation, except in formulations wherein an intended synergistic effect is demonstrated by the claims or examples herein (e.g., a formulation consisting essentially of yohimbine and Naloxone, or pharmaceutically acceptable salts thereof). In another example, a pharmaceutical formulation can consist essentially of yohimbine, meaning that the formulation is provided in the form of a nasal spray, an inhaled formulation, an orally administered formulation, or an injection formulation, each of which is tailored for a fast-acting agent, therapeutic agent, or antidote but not tailored for long-term administration (e.g., as a dietary supplement). In another example, in the case of a preventative supplement to purposefully prevent an overdose, the opposite, long-term supplementation, can be referred to with “consisting essentially of”. This example could be applied to a fentanyl addict who habitually purchases street fentanyl that is likely to be laced with xylazine. The term “consisting essentially of” can also be exemplified by plain language provided in the claims.

The term “statistically significant” or “significantly” refers to statistical significance and generally means a two-standard deviation (2SD) or greater difference.

As used herein, the term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects particularly include human subjects as described herein. The human subject may be a pediatric, adult, or a geriatric subject. The human subject may be of either sex.

As used herein, the terms “effective amount”, “therapeutically effective amount”, and “pharmaceutically effective amount” include an amount sufficient to prevent or ameliorate a manifestation of or a suspected manifestation of a medical condition, such as a drug overdose. The manifestation can be a sign or symptom or otherwise. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the medical condition and on the characteristics of the individual, such as general health, age, sex, body weight and tolerance to drugs. It will also depend on the degree, severity, and type of medical condition. The skilled artisan will be able to determine appropriate dosages depending on these and other factors. Examples of other factors can be route of administration and length of administration(s). The compositions can also be administered in combination with one or more additional therapeutic compounds.

The term “protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed.¹¹ and in Harrison, et al., Compendium of Synthetic Organic Methods, Vols. 1-8.¹² Examples of representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Examples of representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. For example, a compound that prevents epilepsy may reduce the frequency of seizures and/or reduce the severity of seizures.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered at the same time, within one minute, 2 minutes, 4 minutes, 6 minutes, 10 minutes, 30 minutes, or an hour or 90 minutes of one another. Thus, an individual who receives such treatment can benefit from a combined effect of different therapeutic compounds.

As used herein, the terms “treat,” “treatment,” “treating,” or “amelioration” when used in reference to a disease, disorder, or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), sign(s), diminishment of extent of the deficit, stabilized (i.e., not worsening) state of a symptom or condition, delay or slowing of onset of symptoms or indications, and an increased lifespan as compared to that expected in the absence of treatment.

As used herein, the term “long-term” administration means that the therapeutic agent or drug is administered for a period of at least 12 weeks. This includes that the therapeutic agent or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by aerosol inhalation, injection, ingestion, transfusion, implantation, application (e.g., topical, otic, or ocular), or transplantation. Administration can be accomplished by an implant. In some embodiments, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into an artery, vein, lymph node, or organ (e.g., heart).

The terms: “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The term “prodrug” is intended to encompass compounds which, under physiologic conditions, are converted into the therapeutically active agents of the present invention (e.g., a compound such as yohimbine). A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds utilized herein such as yohimbine in a formulation can be replaced with the corresponding suitable prodrug, e.g., wherein a hydroxyl in the parent compound is presented as an ester or a carbonate or carboxylic acid present in the parent compound is presented as an ester. In some embodiments, a “prodrug” is made by using an absorbing particle that subsequently releases an active form after administration.

In some embodiments, the decrease in the one or more signs or symptoms is evaluated according to the DSM-5.¹³ In some embodiments, signs are observed or measured by a health care provider. Symptoms can be reported by the subject. In some embodiments, the decrease of signs or symptoms occurs in less than about 120 minutes, 90 minutes, less than about 60 minutes, less than about 30 minutes, less than about 15 minutes, less than about 10 minutes, or less than about 5 minutes, or less than about 3 minutes, or less than about 1 minute.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, the term “addiction” relates generally to a treatable, chronic medical disease involving complex interactions among brain circuits, genetics, the environment, and an individual's life experiences. People with addiction use substances or engage in behaviors that become compulsive and often continue despite harmful consequences. Substance addiction (or drug addiction) is a neuropsychiatric disorder characterized by a recurring desire to continue taking the drug or drugs despite harmful consequences. Non-substance addiction (or behavioral addiction) covers examples such as pathological gambling, food addiction, internet addiction, and mobile phone addiction.¹⁴ In some embodiments, the technology disclosed herein can be utilized to prevent or to treat addiction. As used herein, a “substance” consumed by a subject and suspected to be or involved in an overdose can be a small molecule less than 1000 MW or a large molecule not less than 1000 MW including biologics, oligonucleotides, peptides, oligosaccharides, and psychoactive large molecules. Any of the antagonists or therapeutic agents disclosed herein can be used as or in combination with small molecules and/or large molecules as discussed herein.

As used herein, a subject may or may not be aware of suffering from an addiction. A health care provider may suspect addiction or may have confirmed addiction.

A subject can be one who has been previously diagnosed with or identified as suffering from or having a condition in need of treatment (e.g., an addiction or related behavior) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. For example, a subject can be one who exhibits one or more risk factors for a condition, or one or more complications related to a condition or a subject who does not exhibit risk factors. A “subject in need” of treatment for a particular condition can be a subject having that condition, diagnosed as having that condition, or at risk of developing that condition. In another example, the subject has been brought into an overdose situation entirely without the subject's knowledge and/or intent. For example, a subject can obviously be in an overdose condition but not be responsive to Naloxone; as described herein the present methods and formulations may save the subject's life.

### Pharmaceutical Compositions

The compositions and methods of the present invention may be utilized to prevent a need for other treatment, to provide benefit when other treatment(s) fail, or to treat an individual in need thereof. In some embodiments, the individual is suspected of needing treatment. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In some embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible compositions employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. Inhalation can include inhalation of a liquid (droplets or aerosol). Inhalation can include a micronized powder adhered to carrier particles or can be without carrier particles. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules, including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymers and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, micro-emulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art. See, e.g., Isselbacher, et al., (1996).¹⁵

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The subject or patient receiving this treatment is any animal in need, including primates, in particular humans; and other mammals such as equines bovine, porcine, sheep, feline, and canine; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, I-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I-tartaric acid, thiocyanic acid, p-toluenesulfonic acid, trifluoroacetic acid, and undecylenic acid salts. As shown below the HCl can be replaced by other pharmaceutically acceptable cation(s) and/or anion(s) or the methyl ester can be removed/cleaved.

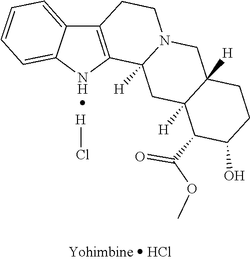

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent. The invention contemplates polymorphs, cocrystals, and amorphous forms of all substances discussed herein. As discussed above, solvates and/or hydrates can be formed by, for example, a slow evaporation whereby water and/or solvent remain hydrogen bonded with OH groups in the molecule. The formation of a solvate/hydrate can be quickly confirmed after the evaporation by using attenuated total reflectance Fourier transform infra-red spectroscopy wherein the solid solvate/hydrate is directly placed on the instrument and the subsequent IR spectrum is compared to the IR spectrum of the solid non-solvate, non-hydrate.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy;¹⁶ The Encyclopedia of Molecular Cell Biology and Molecular Medicine;¹⁷ Molecular Biology and Biotechnology: a Comprehensive Desk Reference;¹⁸ Immunology;¹⁹ Janeway's Immunobiology;²⁰ Lewin's Genes XI;²¹ Molecular Cloning: A Laboratory Manual;²² Basic Methods in Molecular Biology;²³ Laboratory Methods in Enzymology;²⁴ Current Protocols in Molecular Biology (CPMB)²⁵; Current Protocols in Protein Science (CPPS);²⁶ and Current Protocols in Immunology (CPI).²⁷

In the embodiments discussed and in any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

### Yohimbine Rescue in Unidentified Drug Overdose

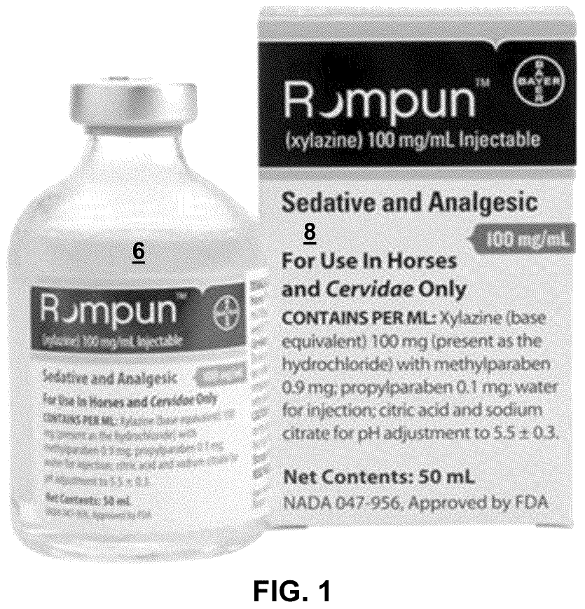

In some embodiments, the invention disclosed herein provides surprising systems, methods, and compositions to address the US national overdose threat.¹ In some embodiments, the invention improves upon the existing technology used in drug overdose emergencies which includes only administration of Naloxone and supportive therapy. A novel approach to reversing otherwise lethal drug overdoses that appear to be opioid induced but are not responsive to Naloxone administration is disclosed herein. There are currently no xylazine antidotes in use for human drug overdoses.^{5,6} Tragically as a result, the number of drug overdose deaths in the United States has been steadily rising since 2019. This situation is a major public health emergency. The invention disclosed herein is life-saving and safe to administer. FIG. 1 demonstrates xylazine is a potent horse tranquilizer, but xylazine is disastrously being combined with fentanyl.¹ New applications and systems using α₂ adrenergic agonists are also described in detail in the following Examples.

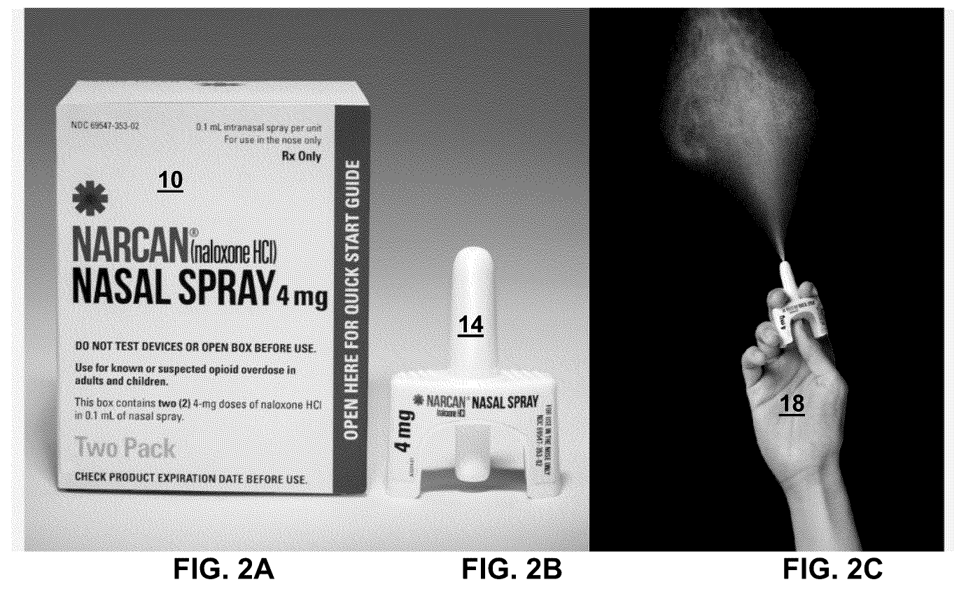

Xylazine, an α₂ adrenergic agonist, is a central nervous system (CNS) depressant which is approved by the FDA for use in tranquilizing animals in veterinary medicine. FIG. 1 provides a photo of xylazine injectable ROMPUN® (Bayer Pharmaceuticals tradename) for FDA-approved use in horses (strictly not for human use). At left is a syringe accessible bottle 6. At right is example packaging 8 showing ingredients of xylazine hydrochloride, methylparaben, propylparaben, water, citric acid/sodium citrate for pH. This drug is frequently found in the illicit drug supply in the United States and has contributed to the drug overdose deaths. It is widely known in the lay press that emergency medical service (EMS) providers increasingly must use multiple doses of Naloxone in individuals suspected of an opioid overdose in attempting to reverse drug effects. Such individuals may not be able to be revived, contributing to drug overdose deaths. FIG. 2 provides an example illustration of an FDA-approved NARCAN® (Naloxone HCl) nasal spray. FIG. 2A provides a photo of the nasal spray packaged in a box 10. FIG. 2B provides a photo of a nasal spray device 14 out of the box. FIG. 2C provides a photo of a hand discharge of the nasal spray from the device 18. The nasal spray (Narcan) has been approved for over the counter sale and distribution as of Mar. 29, 2023.⁷ The technology herein can combine, in some embodiments, Naloxone with yohimbine, making such an inhaler save lives.

Drug overdose deaths have been steadily increasing in the United States for several years concomitant with the increasing presence of xylazine in the drug supply. Further, those sustaining severe and often lethal drug overdose often have ingested other CNS depressants with opioids including alcohol and benzodiazepines.

α₂ adrenergic antagonists are known antidotes to the effects of xylazine in some animal species. Like xylazine, the CNS depressants alcohol and benzodiazepines also suppress noradrenergic function important to cardiac function, respiration, and sedation. According to the methods of the present disclosure, α₂ adrenergic antagonist medications not only reverses the effects not only of xylazine, but also of other drugs, e.g., alcohol and benzodiazepines, that suppress noradrenergic function in humans.

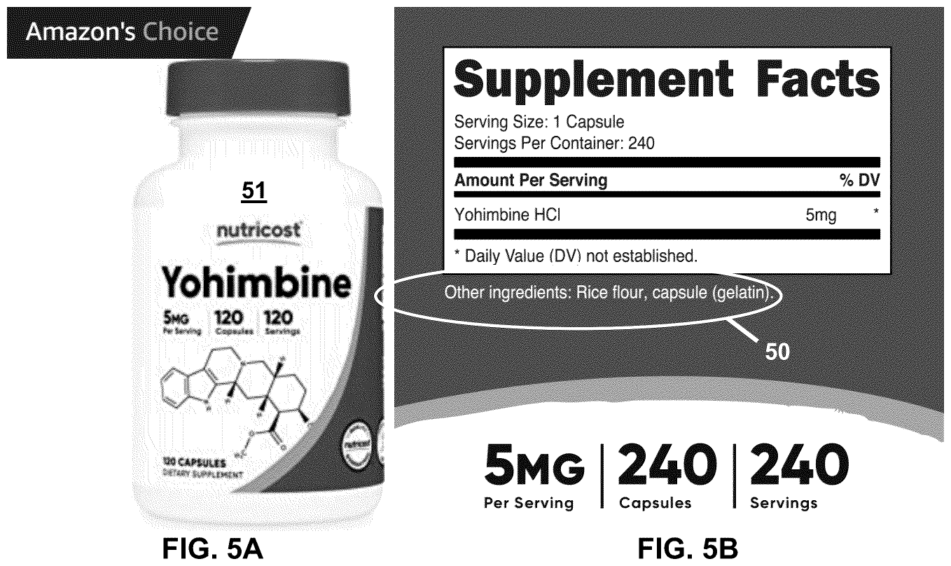

The medication yohimbine has already been approved by the FDA to treat impotence in humans. The doses to be used in drug overdose have been used in research studies thereby providing safety data indicating that the medication may be safely given in an overdose situation. Yohimbine is a generic drug and cost should be relatively low. It is administered without issues and can be combined with Naloxone to make a product that will better address the contributing factors to drug overdose. FIG. 5 provides a photo of NUTRICOST® Yohimbine on sale at Amazon.com.⁸ FIG. 5A shows a bottle 51 containing capsules. FIG. 5B shows the capsules are formulated 50 with rice flour in capsule (gelatin).

The present invention, in one of its embodiments, provides a method for preventing and/or treating a subject undergoing a substance overdose or suspected of undergoing a substance overdose. According to some aspects, the substance comprises one or more drug(s) that are unknown. In some embodiments, the substance comprises a drug of abuse (e.g., heroin, fentanyl, or other opioids), laced with xylazine and xylazine congeners, alcohol, fentanyl/opioids, and benzodiazepines. The symptoms of substance overdose include one or more of dry mouth, drowsiness, hypertension, hypotension, tachycardia, bradycardia, hyperglycemia, reduced heart rate, hypothermia, coma, respiratory depression, and/or dysrhythmia. The term “suspected” can include previously diagnosed of the overdose or previously diagnosed as having a likelihood of the overdose.

In some embodiments, the method comprises an initial administration of a common substance abuse therapeutic (e.g., Naloxone) to the subject. When the initial sole administration of the common substance abuse therapeutic is ineffective in reducing the symptoms of substance overdose, the subject is then administered an α₂ adrenergic antagonist. In alternative embodiments, the common substance abuse therapeutic (e.g., Naloxone) is co-administered with an α₂ adrenergic antagonist. These can be co-administered (i) in separate routes of administration, in the same route of administration, or (iii) in the same formulation. The administration of the α₂ adrenergic antagonist, alone or in combination with a common substance abuse therapeutic, causes a partial or complete reversal of the substance overdose.

According to some aspects, the method is wherein said antagonist is provided in a fast-acting formulation capable of providing antagonist plasma concentration levels about ≥50% of peak concentrations before about 60 minutes, optionally before about 30 minutes, or before about 15 minutes, or before about 10 minutes, or before about 5 minutes, after administration.

In some embodiments, the formulation is provided in a form of a buccal film, sublingual tablet, nasal spray, inhaled aerosol, intravenous injectable, intra-arterial injectable, intracardiac injectable, hydrogel or organogel, powder, intramuscular injectable, subcutaneous injectable, intradermal injectable, eye (ocular) formulation drops, ocular mist, or fast absorbing topical formulation. According to some aspects, the formulation is operative to be administered by a non-health care provider in an emergency or in a non-healthcare (field administration) setting.

In another aspect, the disclosure provides a prophylactic method wherein the α₂ adrenergic antagonist is administered long term to a subject at risk of substance abuse.

In some embodiments, the present invention provides a product for fast-acting use in humans comprising GMP (Good Manufacturing Practice as enforced by the FDA) quality yohimbine or a pharmaceutically acceptable salt thereof, wherein said product is capable of providing a field administration including the administration outside of a healthcare facility. According to some aspects, the product further comprises Naloxone.

In some embodiments, the product disclosed herein is fast-acting, capable of providing yohimbine (or other antagonist) plasma concentration levels≥50% of peak concentrations before 60 minutes after an administration of the product to a human subject. According to some aspects, said product includes a formulation in a form of a buccal film, sublingual tablet, nasal spray, inhaled aerosol, intravenous injectable, intra-arterial injectable, intracardiac injectable, hydrogel or organogel, powder, intramuscular injectable, subcutaneous injectable, intradermal injectable, eye (ocular) formulation drops, ocular mist, or fast absorbing topical formulation (e.g., with dimethyl sulfoxide skin penetrant or nanoparticles/gels).

The product can be configured, according to some aspects, for emergency use in humans. In another example, the product is provided over the counter, with a shelf life (at room temperature or 25° C.) of greater than 6 months, greater than a year, greater than two years, or greater than five years.

In some embodiments, the product disclosed above is wherein the product consists of yohimbine (or the salt thereof) or consists essentially of yohimbine (or the salt thereof) such that only other ingredients are excipients and not an additional API (active pharmaceutical ingredient). According to some aspects, the product consists essentially of yohimbine and Naloxone, such that only other ingredients are excipients and not an additional API.

Depending on the formulation, delivery, mode of administration, and packaging, the product can be about 0.1 mg/mL to about 100 mg/mL yohimbine, about 1 mg/mL to about 50 mg/mL yohimbine, about 5 mg/mL to about 40 mg/mL yohimbine, about 10 mg/mL to about 30 mg/mL yohimbine, or about 20 mg/mL yohimbine configured for intravenous or intramuscular administration with sterile syringes for field administration.

In some embodiments, the present invention provided a combination product including Naloxone and yohimbine operative to be administered via intravenous, intramuscular, or intranasal route in preventing and/or treating drug overdose where the drug(s) ingested are unknown. According to some aspects, the Naloxone and yohimbine are provided in a liquid form.

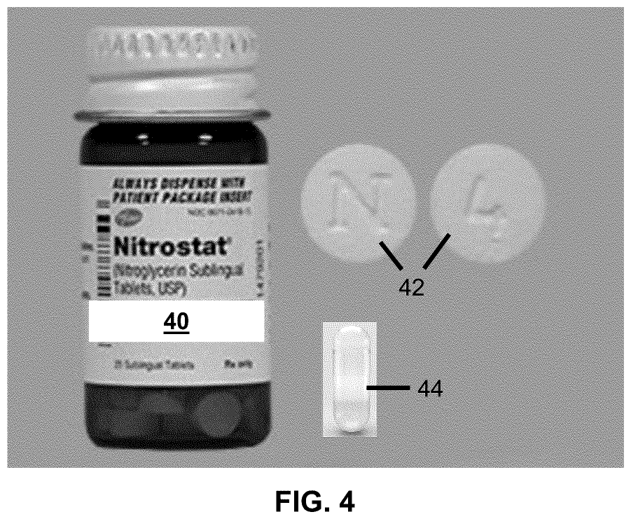

In some embodiments, a deployable product is disclosed including a formulation of an α₂ adrenergic antagonist and/or an antagonist of a receptor of α₂ adrenergic, or a precursor of said antagonist, said formulation provided in a form of a buccal film, sublingual tablet, nasal spray, inhaled aerosol, intravenous injectable, intra-arterial injectable, intracardiac injectable, hydrogel or organogel, powder, intramuscular injectable, subcutaneous injectable, intradermal injectable, eye (ocular) formulation drops, ocular mist, or fast absorbing topical formulation. According to some aspects, the deployable product is wherein the formulation includes yohimbine and optionally Naloxone. FIG. 4 provides a photo of NITROSTAT® (Pfizer Pharmaceuticals tradename) for FDA-approved use in humans as an example of tablets 42, sublingual tablets 42, and/or capsules 44 in a bottle 40. Nitroglycerin is a decades-old sublingual and provides an example of a fast-acting sublingual that can be achieved with the antagonists disclosed herein.

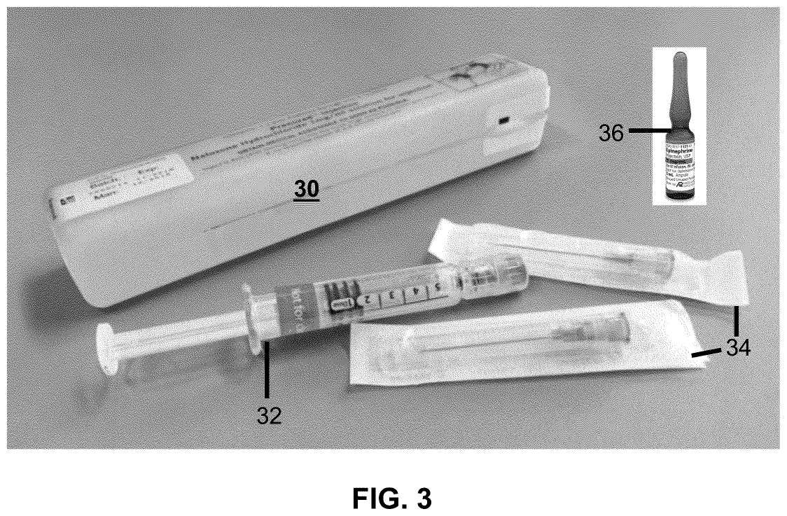

In some embodiments, a deployable kit is disclosed herein including any of the products disclosed above and instructions for use. The instructions can include any step(s) of the methods disclosed herein. FIG. 3 provides an example photo of a kit for providing α₂ adrenergic antagonist and/or an antagonist of a receptor of α₂ adrenergic, or a precursor of said antagonist in an example container 30. The container 30 can contain a nasal spray as depicted in FIG. 2, and/or a syringe 32, needles 34, and ampule 36. The container 30 can contain a bottle 40 as the example in FIG. 4. The ampule 36 can be for repeated use as shown in the accessible bottle in FIG. 1.

In some embodiments, the invention provides a system for preventing substance overdose, likelihood of substance overdose, and/or one or more deaths by substance overdose in a population of humans, said system comprising providing any steps of the method(s) disclosed herein, the yohimbine, the formulation, the deployable product(s), and/or the kit(s), to at least one member of the population.

In some embodiments, a method of diagnosing a human subject for an exposure to xylazine is provided herein, said method comprising the steps of: (1) administering an α₂ adrenergic antagonist and/or an antagonist of a receptor of α₂ adrenergic, or a precursor of said antagonist to the subject; and (2) monitoring one or more signs or symptoms for a modulation/change/reversal in the subject; whereby the presence of the modulation/change/reversal indicates the exposure to xylazine. According to some aspects, the method is wherein said antagonist comprises or consists essentially of yohimbine and/or Naloxone. The method of diagnosing can be performed, according to some aspects, wherein before execution of step (1), the subject has previously had an administration of Naloxone and a monitoring of one or more signs or symptoms for a modulation/change/reversal in the subject; and there was not the presence of said modulation/change/reversal after the administration of the Naloxone. In some embodiments, the “exposure to xylazine” is an unidentified overdose.

In an alternative aspect, the present invention provides a long-term method of preventing a drug overdose in a subject in need thereof, by administering a therapeutically effective amount of an α₂ adrenergic antagonist (e.g., yohimbine).

The invention contemplates that the above-described compounds of yohimbine can be derivatized or can be structurally altered, for example, by addition or substitution of one or more atoms using a radioisotope or using a different element (e.g., B in place of C), by removal of an ester or by addition of a salt form, an amino acid, a sugar, or a peptide. In some embodiments, the above-described compounds are attached to or associated with a targeting moiety. In some embodiments, the targeting moiety is a particle or an antibody with affinity for a specific type of cell.

In some of the various embodiments, the technology disclosed herein can be envisioned by studying the following list of features either alone or in combination with any other aspect discussed herein; any feature can be combined with any other feature:

Feature 1: A method for preventing and/or treating a subject having a substance overdose or suspected of having a substance overdose, the method comprising administering an alpha-2 (α₂) adrenergic antagonist to the subject, whereby one or more signs or symptoms of the substance overdose are prevented or reduced.

Feature 2: The method of feature 1, wherein the α₂ adrenergic antagonist is selected from the group consisting of: a blocker, an orthosteric antagonist, allosteric antagonist, conformation changing binder, a modulator, a partial inverse agonist/inverse agonist, or a combination thereof.

Feature 3: The method of feature 1, wherein the α₂ adrenergic antagonist comprises yohimbine or a pharmaceutically acceptable salt and/or hydrate or solvate thereof.

Feature 4: The method of feature 1, wherein the substance overdose comprises one or more unknown drugs or substances in the subject.

Feature 5: The method of feature 1, wherein the administration of the α₂ adrenergic antagonist results in a partial or complete reversal of symptoms of the substance overdose at least contributed to by one or more drugs included in said substance comprising an opioid or a synthetic opioid, xylazine and xylazine congeners, an alcohol, benzodiazepines, or a combination thereof.

Feature 6: The method of feature 1, wherein the substance comprises fentanyl, heroin, hydromorphone, methadone, buprenorphine; morphine, codeine, xylazine and xylazine congeners, or a combination thereof.

Feature 7: The method of feature 1, wherein the one or more signs or symptoms of the substance overdose are selected from the group consisting of: dry mouth, drowsiness, hypertension and/or hypotension, tachycardia, bradycardia, hyperglycemia, reduced heart rate, hypothermia, coma, respiratory depression, and/or dysrhythmia.

Feature 8: The method of feature 1, wherein said α₂ adrenergic antagonist is administered in a fast-acting formulation capable of providing antagonist plasma concentration levels≥50% of peak concentrations within 60 minutes of the administration.

Feature 9: The method of feature 8, wherein said formulation is provided in a form selected from the group consisting of: a buccal film, sublingual tablet, nasal spray, inhaled aerosol, inhaled solid powder, intravenous injectable, intra-arterial injectable, intracardiac injectable, hydrogel or organogel, powder, intramuscular injectable, subcutaneous injectable, intradermal injectable, eye (ocular) formulation drops, ocular mist, or fast absorbing topical formulation.

Feature 10: The method of feature 1, wherein the α₂ adrenergic antagonist is co-administered with Naloxone.

Feature 11: The method of feature 1, wherein the α₂ adrenergic antagonist is administered after an initial administration of Naloxone to the subject was ineffective in reversing and/or in diminishing the symptoms of substance abuse.

Feature 12: The method of feature 1, wherein the α₂ adrenergic antagonist is administered as a prophylactic in a subject exhibiting one or more signs or symptoms of chronic substance abuse selected from the group consisting of: soft tissue injuries, skin ulcers, abscesses, and/or tissue necrosis.

Feature 13: A lifesaving kit for preventing and/or for treating a subject having a substance overdose or suspected of having a substance overdose, the kit comprising: (1) an alpha-2 (α₂) adrenergic antagonist in a fast-acting pharmaceutical formulation suitable for an emergency administration to the subject; and (2) instructions for use.

Feature 14: The lifesaving kit of feature 13, further comprising an aerosol dispenser device, a particulate dispenser device, an autoinjector device, a sublingual tablet, an eyedropper device, or a combination thereof.

Feature 15: The lifesaving kit of feature 13, wherein the α₂ adrenergic antagonist comprises a blocker, an orthosteric antagonist, allosteric antagonist, conformation changing binder, a modulator, a partial inverse agonist/inverse agonist, or a combination thereof.

Feature 16: The lifesaving kit of feature 13, wherein the α₂ adrenergic antagonist comprises yohimbine or a pharmaceutically acceptable salt and/or hydrate or solvate thereof.

Feature 17: A system for preventing a prevalence or a rise in a number of occurrences of overdoses in a population of subjects, each of the subjects in the population spread over a geographic area, the system comprising: (1) a plurality of kits including an alpha-2 (α₂) adrenergic antagonist in a fast-acting pharmaceutical formulation suitable for an emergency administration to the subject suffering from an overdose or suspected of suffering from an overdose; (2) a deployment of the plurality of kits over the geographical area with a greater number or a greater geographical concentration of each of the kits from the plurality deployed in one or more geographical areas known to have a higher incidence of a drug overdose.

Feature 18: The system of feature 17, wherein the system is further comprising: (3) determining the one or more geographical areas known to have a higher incidence of a drug overdose based upon an emergency ambulance and/or hospital emergency room record for each of the one or more geographical areas; and (4) repeating the deployment of (2) to provide a sharper focus of more of the kits or a greater concentration of the kits in the geographical areas known to have a higher incidence of a drug overdose.

Feature 19: The system of feature 17, further comprising (5) measuring the incidence of a drug overdose based upon an emergency record for each of the one or more geographical areas and determining if an incidence of one or more fatal drug overdoses decreases after the deployment in (2).

One skilled in the art will appreciate that suitable methods of administering the compound of the present invention or composition thereof to a mammal such as a human, are known, and, although more than one route can be used to administer a particular composition, a particular route can provide a more immediate and more effective reaction than another route.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. The methods, kits, formulations, and devices disclosed herein can be combined in any way into systems to address the current public health emergency.

The present invention provides health care systems, deployments, and strategies for use in preventing and/or in treating suspected or established drug overdose emergencies that can be implemented from a policy-wide perspective. For example, the invention provides the use of α₂ adrenergic antagonists as a drug class for use in the reversal of drug overdoses from exposure to unknown street drugs. The drug overdoses can be contributed to by xylazine, xylazine congeners, alcohol, and/or benzodiazepines in humans. In some embodiments, the invention discloses administration of one or more α₂ adrenergic antagonists (e.g., yohimbine) in addition to or in place of a different treatment. Surprisingly, the methods and cures disclosed herein have little to no risk but provide immense and immediate public benefits.

The doses of yohimbine to be used to prevent or treat drug overdose have been used in research studies thereby providing safety data indicating that the yohimbine (or a salt thereof) may be safely given in an overdose situation. As an example, yohimbine is a generic drug and cost can be relatively low. It is administered without issues and can be combined with Naloxone to make a product that will better address the contributing factors to drug overdose. By acting, combining, and formulating as disclosed herein lives can be saved.

The technology described herein is further illustrated by the following examples which in no way should be construed as being further limiting. The Examples are provided to demonstrate examples of future planned work, which in some experiments is emergency work. Although methods and materials similar or equivalent to those described herein can be used in the practice or testing of this disclosure, suitable methods and materials are described below.

### EXAMPLES

The invention now being generally described, it will be more readily understood by reference to the following examples which are included merely for purposes of illustration of certain aspects and embodiments of the present invention and are not intended to limit the invention.

### Example 1 Pharmacokinetics of Yohimbine Formulations for Overdose Reversal: Phases I and II Studies

Due to long-established safety profiles of yohimbine, at least partial waiver of full Ph. I is requested. Focus on rapidly absorbed (fast-acting) α₂ adrenergic antagonists are tested using real time data acquisition. For example, rapidly absorbed formulations including yohimbine are compared to determine if an intranasal (i.n.) spray dose provides early absorption similar to intramuscular (i.m.) injection. To be effective, the intranasal (i.n.) spray dose must be adequate but not excessive, and early absorption must be comparable to i.m. injection. The pharmacokinetics (PK) of a specially produced concentrated novel nasal spray are investigated. Formulations of the nasal spray begin by targeting about a 4 mg dose of yohimbine hydrochloride in about a 0.1 mL intranasal spray. For example, the formulation can include inactive ingredients of benzalkonium chloride (preservative), disodium ethylenediaminetetraacetate (stabilizer), sodium chloride, base or hydrochloric acid to adjust pH, and purified water. In another example, the formulation is preservative free and sterile filtered or irradiated. The specific aims are to: (1) estimate PK profiles of i.n. Naloxone, (2) compare early systemic exposure with i.n. versus i.m. Naloxone and (3) estimate i.n. bioavailability.²⁸ Dose escalation and lower dosing schedules are also investigated. Fast-acting (emergency) formulations are developed to provide antagonist(s) in plasma concentration levels ≥50% of peak concentrations within 1 minute after administration, within 2 minutes after administration, within 3 minutes after administration, within 4 minutes after administration, within 5 minutes after administration, within 10 minutes after administration, within 15 minutes after administration, within 30 minutes after administration, within 60 minutes after administration, and within 90 minutes after administration.

Each formulation is ranked according to speed or how fast-acting it can be. The formulations are next tested along with presence of Naloxone.

Depending on the formulations, mode of administration, and delivery/packaging, the product can be about 0.1 mg/mL to about 100 mg/mL yohimbine, about 1 mg/mL to about 50 mg/mL yohimbine, about 5 mg/mL to about 40 mg/mL yohimbine, or about 10 mg/mL to about 30 mg/mL yohimbine, and any forms of fast-acting administration are investigated. Formulations are implemented in Ph. II further below.

### Example 2 Yohimbine Reversal of Xylazine Sedation in Humans: Phases I and II Studies

Yohimbine is currently sold over the counter (OTC) in the US, (e.g., FIG. 5). Pharmaceutical (i.e., prescription) level quantities of yohimbine are found in OTC dietary supplements in the US.²⁹ In some previous human studies, on average, an oral dose of 5-15 mg yohimbine (HCl salt) causes a maximum therapeutic whole blood level in the range of about 40-500 ng/mL.³⁰ Yohimbine has been tested in a variety of clinical trials³¹ such as posttraumatic stress disorder,³² panic disorder,³³ emotional stress response,³⁴ pteromerhanophobia (fear of flying),^{3I} and cancer.³⁶ As discussed in Example 1 above, the present technology provides fast-acting and combination formulations. Ph. II is applied for under lifesaving conditions.

### Ethical Considerations

Emergency personnel are present. Xylazine is not approved for use in humans at the federal level although several states (including Rhode Island) have placed this drug on Schedule Ill which will make research studies in humans with this drug possible (FIG. 1). Xylazine was first synthesized in 1962 by Bayer Pharmaceuticals and was investigated for potential human use in clinical trials as an analgesic, sleeping aid, and anesthetic. However, these trials were terminated due to its severe hypotension and central nervous system (CNS) depressant effects.³⁷ Emergency Use Authorization is sought with the FDA for administration in life threatened, emergency situations, in particular, where there is a national emergency,¹ and where Naloxone is not effective to save lives.⁶ Emergency Use Authorization, optionally with preliminary animal data below, is sought with the FDA to proceed with administration of about 0.01-0.15 mg/kg xylazine to healthy subjects. The level of xylazine sedation is evaluated based on the following scoring: 0, no effect; 1, light sedation (affected behavior and movement, slower reactions but mobile); 2, moderate sedation (depressed behavior, minor response to handling); and 3, deep sedation (unaware of their surroundings). The following example illustrates testing xylazine sedation with and without administration of yohimbine.

After xylazine administration, some physiological variables that are recorded are pupil dilation, respiratory rate (breaths/min), blood pressure, pulse rate, and temperature. Most variables are monitored from wrist monitors and are continuously recorded after xylazine administration. If available electrocardiogram (ECG or EKG) and/or brain activity is recorded. Urine samples and arterial blood samples are collected from some subjects for analysis of pulmonary gas exchange, acid-base balance, and ADMET (absorption, distribution, metabolism, and excretion).

For reversal of the effects of xylazine, yohimbine hydrochloride is administered by rapid injection in an auricular vein at about <1 mg per 10 mg xylazine (about 0.001-0.15 mg/kg), which can equal a total dose of about 2-20 mg. Nasal administration is also accomplished with saline solution of yohimbine at first, with other formulations quickly made. The time from xylazine injection to administration of yohimbine is recorded. The time for reversal is decided during each immobilization, depending on continuously recorded data (sampling procedures) and statistical analyses. The times from yohimbine injection until first sign of recovery and, when possible, to full recovery are recorded. Re-sedation may be investigated after using yohimbine as a reversal.

For using fentanyl, female Sprague-Dawley rats are randomly divided in groups, 6 rats/group. Three control groups include a group with no treatment, a group with fentanyl and xylazine treatment, and a group with xylazine treatment. Three other groups are administered low dose yohimbine, mid dose yohimbine, and high dose yohimbine. Three separate groups are administered low dose yohimbine w/Naloxone, mid dose yohimbine w/Naloxone, and high dose yohimbine w/Naloxone. Additional groups are administered fentanyl with xylazine or xylazine and low, mid, and high dose yohimbine and yohimbine with Naloxone. If Emergency Use Authorization is granted, human subjects otherwise non-responsive to Naloxone are administered yohimbine.

### Example 3 Virtual (in Silico) Screening of Alpha-2 (α₂) Receptor Antagonists

The 3D structure of human α_{c} adrenergic receptors are retrieved from a protein data bank. The protein structure(s) is/are optimized and prepared for in silico screening or docking of ligands.

After minimization, the protein structure is visualized. Regions capable of accommodating ligands or small molecules are identified.

A list of potential ligands (example in Table 1)³⁸ is retrieved and the structures are optimized in silico. Solvent modeling is included wherein suspected dimerization, hydrogen bonding to solvent, or conformational considerations could occur. The ligands are docked to the protein structure and scored for high docking scores.

TABLE 1
Example of α₂ Antagonists

| Drug: | Drug Description: |
|---|---|
| ORM-12741 | ORM-12741 has been used in trials studying the basic science and treatment |
| (Orion, Finland) | of Alzheimer's Disease. |
| Idazoxan | Idazoxan has been used in trials studying the basic science of Molecular |
| | Imaging, Alzheimer Disease, and Major Depressive Disorder. |
| Yohimbine | An α₂-adrenergic blocker and sympatholytic found in supplements. |
| Lurasidone | An atypical antipsychotic used to treat schizophrenia and depressive episodes |
| | associated with bipolar I disorder. |
| Mirtazapine | A tetracyclic antidepressant used in the treatment of major depression and is |
| | used off-label as a drug for insomnia and to increase appetite. |
| Esmirtazapine | Investigated for use/treatment in insomnia and sleep disorders. |

Alpha-2 antagonists are cross referenced against known activities at other receptors, such as 5-hydroxytryptamine (5-HT) receptors, or serotonin receptors, as well as for any known metabolites. An example is shown in Table 2.³⁸

TABLE 2
Example Cross Target Considerations

| Drug: | Example Target/Type Consideration: |
|---|---|
| Yohimbine | Alpha-2A adrenergic receptor/target |
| Yohimbine | Alpha-2B adrenergic receptor/target |
| Yohimbine | Alpha-2C adrenergic receptor/target |
| Yohimbine | 5-hydroxytryptamine receptor 1A/target |
| Yohimbine | 5-hydroxytryptamine receptor 1B/target |
| Yohimbine | 5-hydroxytryptamine receptor 1D/target |
| Yohimbine | Dopamine D2 receptor/target |
| Yohimbine | Dopamine D3 receptor/target |
| Yohimbine | 5-hydroxytryptamine receptor 2A/target |
| Yohimbine | 5-hydroxytryptamine receptor 2C/target |
| Yohimbine | Cytochrome P450 2D6/enzyme |
| Yohimbine | Cytochrome P450 3A4/enzyme |
| Yohimbine | ATP-sensitive potassium channel/target |
| Yohimbine | 5-hydroxytryptamine receptor 2B/target |
| Lurasidone | Cytochrome P450 3A4/enzyme |
| Lurasidone | Dopamine D2 receptor/target |
| Lurasidone | 5-hydroxytryptamine receptor 2A/target |
| Lurasidone | 5-hydroxytryptamine receptor 7/target |
| Lurasidone | 5-hydroxytryptamine receptor 1A/target |
| Lurasidone | Alpha-2C adrenergic receptor/target |
| Lurasidone | Alpha-2A adrenergic receptor/target |
| Mirtazapine | 5-hydroxytryptamine receptor 2A/target |
| Mirtazapine | 5HT3 serotonin receptor/target |
| Mirtazapine | Alpha-2A adrenergic receptor/target |
| Mirtazapine | 5-hydroxytryptamine receptor 2C/target |
| Mirtazapine | Kappa-type opioid receptor/target |
| Mirtazapine | Histamine H1 receptor/target |
| Mirtazapine | Cytochrome P450 1A2/enzyme |
| Mirtazapine | Cytochrome P450 3A4/enzyme |
| Mirtazapine | Cytochrome P450 2D6/enzyme |
| Mirtazapine | Alpha-1 adrenergic receptors/target |
| Mirtazapine | Sodium-dependent serotonin transporter/transporter |
| Esmirtazapine | Cytochrome P450 2D6/enzyme |
| Esmirtazapine | Histamine H1 receptor/target |
| Esmirtazapine | 5-hydroxytryptamine receptor 2/target |
| Esmirtazapine | Alpha-2 adrenergic receptors/target |

### Example 4 Prodrug and Precursor Development

Chemical derivatives and mixtures of the antagonists are developed as precursors (or prodrugs). The prodrug can be converted (to active) by an enzymatic activity of the host subject, a release from a carrier, a digestion such as by an amylase, or can be converted by a hydrolysis, wherein a hydroxyl in the parent compound is presented as an ester or a carbonate or carboxylic acid present in the parent compound is presented as an ester. Particles (such as silica) or even a food may be used as carriers/nanocarriers of the therapeutic agent(s) for prodrug/precursor experiments. A precursor or prodrug can be metabolized to the active parent compound (therapeutic agent) in vivo (e.g., the ester is hydrolyzed to the corresponding hydroxyl, or carboxylic acid). Formulation of a precursor/prodrug is accomplished by mixing the subject antagonist with a carrier/nanocarrier, by mixing with an acid and/or an alcohol (then testing hydrolysis at various pH), or by attaching an enzymatically cleavable functional group. The design of prodrugs requires consideration of the following: (1) can the therapeutic agent be modified, (2) what effect will the modification have on the ADMET (absorption, distribution, metabolism, excretion, and toxicity), and (3) can the parent therapeutic agent be regenerated efficiently without producing toxic by-products. Chemical modification of the proposed antagonist requires a suitable functional group that allows reaction to form the targeted pro-moiety that bestows the desired ability for hydrolysis back to the therapeutic agent. Example groups include hydroxyls, amines, carboxylic acids, esters, thiols, and carbonyls. The purpose of the targeted pro-moiety generally dictates its targeted functions in formulation. For example, increased membrane permeability of a hydrophilic drug can be accomplished through lipidation (decreasing hydrophilicity), typically via ester bond formation. In doing so, the prodrug offers improved bioavailability through enhanced absorption from the gastrointestinal tract into systemic circulation or via topical application. Other routes to improve ADMET properties include the addition of ionizable groups to increase solubility (increasing hydrophilicity). The masking of metabolically labile groups is also investigated to prevent their premature breakdown. Future work contemplates the conjugation of peptidic epitopes for the active targeting of specific tissue/cell surface receptors and the site-sensitive activation of the precursor/prodrug for selective release.

Yohimbine is further examined without the ester (in a charged form) to determine if blood brain barrier (BBB) penetration can be controlled using either de-esterification, saponification, charge, or a prodrug as described above.

### Example 5 Example Statistical Analysis

Data are assessed for normality visually through the visual software such as the Quantile-Quantile plot in GraphPad Prism 2020 (GraphPad Software, Inc, La Jolla, CA 92037, USA). A paired t-test is done in EXCEL® 2021 to compare physiological values at 30 min, 45 min, 60 min, 90 min, and later times after xylazine injection. A P-value, 0.05 is considered statistically significant. Descriptive statistics for physiological variables, handling, and response times are presented as mean 6 standard deviations, and median (range, i.e., min-max).

### Example 6 Development of an Antidote for Fatal Xylazine-Laced Overdose

Background: Overdose deaths are increasing despite efforts by governments to address illicit drug use and implement public health approaches. A significant reason for this is due to the increasing prevalence of xylazine (tranq), an α-2 adrenergic agonist and central nervous system depressant, in the fentanyl drug supply and both drugs are increasingly present in illicit stimulants. Xylazine is currently used as an animal tranquilizer. Yohimbine, an α-2 adrenergic antagonist, reverses the effects of xylazine in animals. Yohimbine will be developed as an antidote to xylazine in humans in a form to be administered emergently in overdose situations to reverse cardiovascular and neurological effects of xylazine. Naloxone, the opioid overdose antidote, does not reverse xylazine effects and produces painful withdrawal on administration in overdose. Yohimbine may reverse xylazine effects and substitute for naloxone in some overdose settings making it a product that potentially exceeds the current market for drug overdose antidotes.

Unmet need: Opioid-related overdose deaths are increasing in the U.S. despite a large effort by government to address illicit drug use and to implement public health approaches. A significant reason for this is due to the increasing prevalence of xylazine (aka: tranq) in the drug supply. Xylazine is added to illicit fentanyl, heroin, and cocaine to either enhance drug effects and/or increase drug value by increasing weight.³⁹ According to Centers for Disease Control and Prevention (CDC), a survey of 10 large U.S. cities showed xylazine was present in 1% of overdose deaths in 2015 and 7% in 2020. In 2019, 31% of overdose deaths in Philadelphia included xylazine.⁴⁰ According to surveillance data from the Drug Enforcement Administration (DEA), the prevalence of xylazine in the illicit drug supply continues to grow. Between 2020 and 2021, xylazine has now been identified in all four United States regions (Northeast, South, Midwest, West) with the Northeast having the highest prevalence over the two years reported. DEA Intelligence Program showed that in Forensic laboratory identifications of xylazine increased by 61% in the Northeast, 193% in the South, 7% in the Midwest, and 112% in the West and these are likely underestimates because many jurisdictions do not test for xylazine.⁴⁰ It is expected that these numbers will continue to increase because xylazine is inexpensive ($6-20/kg from Chinese suppliers) and so is a good filler for street drug samples⁴⁰ and because many of those with addiction like the effects of xylazine with some stating that it makes fentanyl ‘more like heroin’ and helps to extend the high of the drugs being used, albeit also expressing concern for the adverse effects of xylazine, principally necrotic skin wounds.⁴¹ Most recently, in 2022, CDC and DEA report that xylazine has now been identified in the illicit drug supply in 48 states and that 23% of fentanyl powder contained this substance as well as 7% of illicit fentanyl pills.⁴⁰ Xylazine depresses noradrenergic function and may act synergistically with potent opioids such as fentanyl to depress respiration and cardiac function. The technology described herein utilizes yohimbine, known in veterinary medicine as an antidote to xylazine, to be used emergently in human overdose situations.

Xylazine, an α-2 adrenergic agonist, is a central nervous system (CNS) depressant which is FDA approved for use in tranquilizing large animals in veterinary medicine.⁴² The technology described herein makes use of α-2 adrenergic antagonists, specifically utilizing yohimbine for which an oral formulation is already FDA-approved for use in humans as a treatment for male erectile dysfunction, but is not approved for use in humans in a form that could be safely administered to an unconscious person. Similar to the existing opioid overdose antidote, naloxone, yohimbine would be administered via intramuscular or intravenous injection or by nasal spray (novel administration route), alone or in combination with naloxone given the high rates of illicit fentanyl containing xylazine and contributing to multidrug overdose. The intranasal administration is in line with the formulation of too many medications that require rapid onset of action including naloxone (opioid reversal), midazolam (agitation, seizure), ketamine (agitation, analgesia, depression) and fentanyl (analgesia).

This intervention improves upon existing technology used in drug overdose emergencies which include only administration of naloxone and supportive therapy. Naloxone, an opioid antagonist medication, has no effect on xylazine toxicity because xylazine depresses adrenergic function to reduce respiratory and cardiac function via adrenergic antagonism.^{42,43} Further, naloxone administration to a person physically dependent on opioids produces painful withdrawal and often, agitation and combativeness following administration that can be difficult to manage by rescuers.⁴⁴ As a result, many are reluctant to administer naloxone and prefer to try to support an overdose victim (with oxygen if available) rather than reverse the overdose (E. McCance-Katz, personal communication with safe consumption site staff). Yohimbine will not produce a withdrawal syndrome and may provide enough sympathetic stimulation to assist in resuscitation making it more acceptable to patients and those administering medication for an unknown drug overdose in the field. Yohimbine can potentially become the preferred overdose antidote for first administration with naloxone administered as a back-up medication should yohimbine not increase respiration.

Yohimbine has been used in human research volunteers extensively by the investigators at doses that are documented to reverse xylazine effects in large animals.⁴⁵⁻⁴⁷ The safety of yohimbine administration in humans at equivalent doses to those used in veterinary medicine to counter anesthetic effects of xylazine has been demonstrated in numerous studies conducted by the investigators. Safety data is summarized below. See, Preliminary Data section below. Research volunteers have included individuals with alcohol (NCT02243709) and opioid use (NCT04051619) disorders, i.e.: some of the population most likely to be at risk of an overdose that could involve xylazine. Therefore, data indicating that yohimbine may be safely given to humans with exposure to illicit drugs has already been collected, analyzed, and exists in the published scientific literature.^{48,49} Such data speaks to the safety of administration in a life threatening overdose situation. Yohimbine is a generic drug and cost to produce a liquid formulation would be relatively low. Yohimbine is easily administered and can be combined with naloxone to make a product that will better address the contributing factors to drug overdose which is a planned product for this project over time. Because of the existence of an FDA-approved medication known already as a xylazine antidote in animals anesthetized with xylazine and because we have extensive safety data on yohimbine administration in humans, rapid development of this product can take place. Rising overdose deaths contributed to by xylazine underscore the urgency of development of this product line.

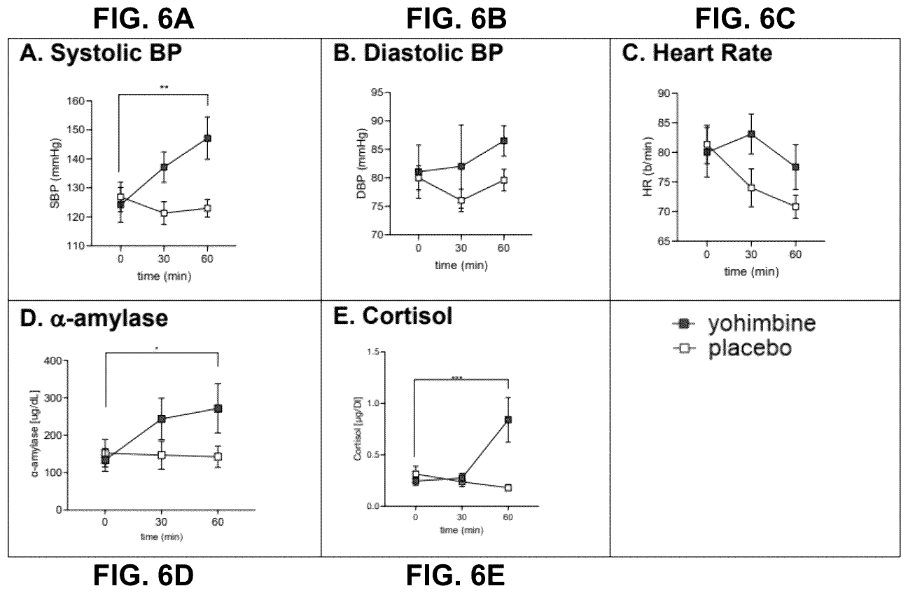

Preliminary Data: Yohimbine safety in addictive disorders and experience of the research team. Yohimbine has been shown to increase sympathetic nervous system activity,^{50,51} has been widely exploited in preclinical research to induce acute noradrenergic activation in laboratory animals under a variety of behavioral paradigms,^{52,53} and in preclinical alcohol use disorder (AUD) models.⁵³⁻⁵⁵ Importantly, yohimbine has been shown to increase stress symptoms^{5,657} and peripheral and central noradrenergic activity in human laboratory studies.^{49,58} In a systematic review,⁴⁸ we showed that yohimbine is a valuable pharmacological probe that has the advantage of being applicable to both preclinical and clinical research settings for addictive disorders. We have safely administered 32.4 mg of yohimbine orally in individuals with alcohol (NCT02243709) and opioid (NCTO4051619) use disorder. Yohimbine 32.4 mg dosing is based on multi-dose administration to heroin-dependent/buprenorphine stabilized volunteers.⁵⁹ Two Investigational New Drug Applications (INDs) have already obtained from the United States Food & Drug Administration (FDA) for the use of yohimbine in individuals with opioid use disorder (OUD) [IND135570] and AUD [IND: 121984]49 with no serious adverse events (SAEs) and no unexpected side effects, untoward reactions or newly recognized risks. To further support the safe use of yohimbine in patients with OUD, preliminary data from a recently concluded clinical trial (NCTO4051619) showed that yohimbine, compared to placebo, moderately increased blood pressure (systolic: p<0.05), heart rate (p=0.078), α-amylase a biomarker sensitive to changes in adrenergic activation (p<0.05), and cortisol level (p<0.01) (FIGS. 6A-6E). FIG. 6A, FIG. 6B, FIG. 6C, FIG. 6D, and FIG. 6E show effects of yohimbine (32.4 g) compared to placebo in the laboratory in patients with opioid use disorder. FIG. 6A shows systolic BP. FIG. 6B shows diastolic BP. FIG. 6C shows a plot of heart rate v. time. FIG. 6D shows α-amylase (μg/dL) v. time. FIG. 6E shows cortisol (μg/dL) v. time. Results from other studies further support the safe use of yohimbine in doses up to 60 mg in humans, including in individuals with OUD⁵⁹, tobacco use disorder⁶⁰, and AUD.⁴⁹

Present study: The overarching goal of the present study is to show that yohimbine can be used to reverse unknown drug overdose that includes contribution to depressant effects by xylazine. A two-stage proof-of-concept research program is conducted to show that yohimbine reverses cardiovascular, respiratory depressant, and neurocognitive effects of xylazine, first under controlled laboratory conditions and then in the field in actual overdose events. The initial stage is a clinical pharmacology study conducted in the Clinical Neuroscience Laboratory at Brown University School of Public Health which: (1) establishes pharmacokinetics of xylazine in humans; (2) determines direct cardiovascular, respiratory and neurocognitive effects of xylazine in humans; and (3) determines whether yohimbine reverses depressant effects of xylazine in healthy subjects. This initial stage provides important data that is foundational to the second stage, which examines the effectiveness of yohimbine in drug overdose in the field. The double-blind, pharmacokinetic drug interaction study in the first stage, occurs prior to administering yohimbine to humans engaged in injection, nasal or smoked illicit drug use who experience symptoms of overdose. In the second stage, yohimbine is utilized in the field. First responders utilize established protocols for drug overdose resuscitation and use yohimbine when there is not a response to naloxone or the person's life is assessed to be in danger. Yohimbine solutions (10 mg/mL), within a dose range for which there is safety data available, are provided to first responders who administer as the clinical situation warrants. The potential benefits outweigh the risks of administration of an FDA medication approved for use in humans that may reverse the effects of xylazine toxicity and assist with resuscitation through yohimbine's cardiovascular and respiratory effects. Overdose resuscitation in the context of lack of adverse responses to yohimbine administration as compared to those observed for naloxone in opioid dependence would potentially result in replacement of naloxone with yohimbine as the first medication of choice in an unknown drug overdose situation. Additional details of the two stages are provided below.

In summary, the current knowledge about the medical use of xylazine comes from its use in animals as an anesthetic agent. The knowledge about the use of xylazine in humans is based on toxicology reports from drug users and drug overdose decedents. The present study provides the first data on the effect of yohimbine on volunteers administered xylazine and establishes that yohimbine is an effective medication for drug overdose reversal. Yohimbine does not produce an opioid withdrawal syndrome on administration, making it safer both for the affected individual and for the rescuer. Further, the noradrenergic stimulant effect of yohimbine is potentially useful as an antidote in other overdose situations involving alcohol and benzodiazepines or ketamine, thus expanding its utility in medical emergencies in the field. Importantly, the investigators have extensive safety data on yohimbine administration to humans at doses used in the present study⁴⁹ (NCT04051619) underscoring feasibility and benefit that would far outweigh risk of yohimbine administration in unknown drug overdose situations.

Market size: The significance of the ongoing and growing problem of drug overdose, and the importance of having antidotes to reverse those toxic and lethal effects of illicit drugs is underscored by the monetary value and expected growth in this market. According to Globe Newswire (4/23), the value of the naloxone (the currently available opioid overdose antidote) market was $320.2 million in 2021 with an expected value of $743.25 million by 2028, inclusive of a predicted 11.1% increase in annual growth.⁶¹ Part of the value of this product lies in the fact that the xylazine antidote, yohimbine, has been FDA approved for years and has been available for human use in capsule (oral) form, while the liquid (injectable) formulation of yohimbine has been extensively used in veterinary medicine.

Further, there is a clinical rationale for the effectiveness of yohimbine in overdoses involving alcohol⁶² or benzodiazepines⁶³ which also reduce cardiovascular integrity and act as respiratory depressants in overdose. Ketamine, increasingly misused as well as an unregulated psychotropic, also reduces cardiovascular and respiratory function in overdose.⁶⁴ Yohimbine acts to reverse those effects, thus representing a pharmacological intervention to unknown overdoses in general.⁶⁵⁻⁶⁷

The cost of drug overdose in the United States is $1 trillion annually.⁶⁸ Over 1 million have died of drug overdose since 1999 in the U.S., with 106,699 overdose deaths in 2021.⁶⁹ The development of yohimbine as an alternative to naloxone with fewer adverse side effects than naloxone and which can be used in any overdose situation characterized by decreased respiration, hypotension and bradycardia will be an opportunity to capitalize on the existing market and need.

Differentiation: The technology described herein offers a major advantage over the only other FDA approved overdose antidote, naloxone, which is only effective in opioid overdose and produces painful withdrawal on administration in physical dependence. Specifically, yohimbine with its pharmacologic action of stimulation of noradrenergic function serves to increase respiratory and cardiovascular activity inhibited by the increasingly prevalent street drug, xylazine, but without producing withdrawal.

Stage 1: Human Laboratory Study of Xylazine and Yohimbine Administration (N=8). The stage 1 study is conducted using a protocol that has been designed based on a published study conducted in sheep with weights from 58-69 kg (similar to adult human weights).⁷⁰ In that study, significant sedation/decreased respiration and decreased blood pressure/heart rate was noted with a dose of xylazine 0.15 mg/kg, which was reversed with yohimbine. To better address safety of volunteers, a dose of xylazine is used, which is 1/3 that administered in the referent study of 0.05 mg/kg, not to exceed (NTE) 5 mg. Data collection is based on published pharmacokinetics of xylazine⁷¹ and yohimbine.^{66,72}

Day 1: Following baseline data collection, 0.05 mg/kg xylazine is diluted in 10 mL sterile normal saline and is administered over 1 minute (min). Blood samples (5 mL) are collected at 0, 5, 15 min and tested for xylazine pharmacokinetics, and vital signs, oxygen saturation, and behavioral responses are collected from the subject. At 20 min post xylazine administration, baseline yohimbine blood level, vital signs, oxygen saturation and behavioral responses are obtained and 0.4 mg/kg yohimbine (40 mg diluted in 10 mL of sterile saline IV) or placebo (10 mL sterile saline) are administered over 1 minute followed by sampling and data collection at timepoints 25 min, 30 min, 40 min, 50 min, 2 hours and 3 hours.

Day 2: Xylazine (0.05 mg/kg, NTE 5 mg) is administered to the subject followed by yohimbine 0.4 mg/kg or placebo (random assignment) at 20 min post xylazine administration using same procedure as for Day 1. Subjects are monitored until return to pre-drug administration baseline and then discharged from the laboratory. At conclusion of the Day 2 session, a blood sample is collected from each subject for hepatic, renal, glucose, and electrolyte indices.

Stage 2: Field study of yohimbine administration in drug overdose (N=8). Staff at safe consumption site(s) in Rhode Island are supplied with yohimbine 10 mg/mL (20 doses) and participate in a training session aimed at providing them with an understanding of the underlying premise for this intervention, which is to provide a medication to individuals determined in the field to be experiencing an opioid/xylazine drug overdose and need emergency intervention. Individuals self-administering drugs containing xylazine as determined by xylazine test strips at these sites will give informed consent for yohimbine administration in the case of overdose. Trained staff administer 0.4 mg/kg yohimbine (based on estimated patient weight: 40 mg) via the IV or IM route if overdose occurs and may repeat the dose once if there is no response in two minutes. Data collection forms is completed to record the primary outcomes: vital signs (respirations, heart rate, blood pressure, oxygen saturation) and mental status prior to (at determination of overdose) and at 2 and 5 minutes following yohimbine administration. If a second dose of yohimbine is administered, vital signs and mental status are collected at 7 and 10 minutes. Time of naloxone administration is also collected. Participating field staff are contacted by one of the investigators to discuss yohimbine administration and response(s) observed.

### REFERENCES

^{1.} The White House. Biden-Harris Administration Designates Fentanyl Combined with Xylazine as an Emerging Threat to the United States. https://www.whitehouse.gov/ondcp/briefing-room/2023/04/12/biden-harris-administration-designates-fentanyl-combined-with-xylazine-as-an-emerging-threat-to-the-united-states/. Accessed Jun. 8, 2023.
^{2.} DEA. PUBLIC SAFETY ALERT: DEA Reports Widespread Threat of Fentanyl Mixed with Xylazine. https://www.dea.gov/alert/dea-reports-widespread-threat-fentanyl-mixed-xylazine. Accessed 13 Jun. 2024.
^{3.} Reyes J. C., et al. The emerging of xylazine as a new drug of abuse and its health consequences among drug users in Puerto Rico. J Urban Health. 2012; 89(3):519-526.
^{4.} Torruella R. A. Xylazine (veterinary sedative) use in Puerto Rico. Subst Abuse Treat Prev Policy. 2011; 6:7.
^{5.} Report DEA Joint Intelligence. The Growing Threat of Xylazine and its Mixture with Illicit Drugs. US Department of Justice DEA. https://www.dea.gov/sites/default/files/2022-12/The%20Growing%20Threat%200f%20Xylazine%20and%20its%20Mixture%20with%20lllicit%20Drugs.pdf. Accessed Jun. 8, 2023.
^{6.} DEA. PUBLIC SAFETY ALERT DEA Reports Widespread Threat of Fentanyl Mixed with Xylazine. https://www.dea.gov/alert/dea-reports-widespread-threat-fentanyl-mixed-xylazine #:˜:text=Xylazine%20and%20fentanyl%20drug%20mixtures,does%20not%20reverse%20its%20effects. Accessed Jun. 5, 2023.
^{7.} FDA. FDA Approves First Over-the-Counter Naloxone Nasal Spray. https://www.fda.gov/news-events/press-announcements/fda-approves-first-over-counter-naloxone-nasal-spray. Accessed Jun. 6, 2023.
^{8.} Amazon. Nutricost Yohimbine. https://www.amazon.com/s?k=nutricost+yohimbine. Accessed Jun. 6, 2023.
^{9.} IUPAC. International Union of Pure and Applied Chemistry GoldBook. https://goldbook.iupac.org/.
^{10.} Merriam-Webster's Online Dictionary. https://www.merriam-webster.com/.
^{11.} Wuts Theodora W. Greene and Peter G. M. Protective Groups in Organic Synthesis. Third Edition. John Wiley & Sons; 1999, (0-471-16019-9).
^{12.} lan T. Harrison Shuyen Harrison. Compendium of Synthetic Organic Methods, Vols. 1-8. John Wiley & Sons; 1971-1996, (978-0-471-35550-2).
^{13.} Association American Psychiatric. Diagnostic and Statistical Manual of Mental Disorders, Fifth Edition. American Psychiatric Association; 2013,
^{14.} Zou Z Wang H, d'Oleire Uquillas F, Wang X, Ding J, Chen H. Definition of Substance and Non-substance Addiction. Adv Exp Med Biol. 2017; 1010:21-40.
^{15.} Isselbacher Kurt J., et al. Harrison's Principles of Internal Medicine, 13th Edition. Shock. 1996; 5(1):78.
^{16.} Porter R. S., & Kaplan, J. L. (Eds.). The Merck manual of diagnosis and therapy (19th ed.). Merck Sharp & Dohme Corp. 2011, (978-0-911910-19-3).
^{17.} Robert S. Porter et al., (eds.). The Encyclopedia of Molecular Cell Biology and Molecular Medicine. Blackwell Science Ltd.; 1999-2012, (9783527600908).
^{18.} Robert A. Meyers (ed.). Molecular Biology and Biotechnology: A Comprehensive Desk Reference. VCH Publishers, Inc.; 1995, (1-56081-569-8).
^{19.} Luttmann Werner. Immunology. Elsevier; 2006,
^{20.} Kenneth Murphy Allan Mowat, Casey Weaver (eds.). Janeway's Immunobiology. Taylor & Francis Limited; 2014, (9780815345305).
^{21.} Krebs Jocelyn E., et al. Lewin's genes XI. 11th ed. Burlington, Mass.: Jones & Bartlett Learning; 2014, (1449659055).
^{22.} Green Michael R. Molecular cloning: a laboratory manual/Michael R. Green, Joseph Sambrook. Cold Spring Harbor, N.Y: Cold Spring Harbor Laboratory Press; 2012, (1936113414).
^{23.} Davis et al. Basic Methods in Molecular Biology. Elsevier Science Publishing, Inc.; 2012, (044460149X).
^{24.} Jon Lorsch (ed.). Laboratory Methods in Enzymology: DNA. Elsevier; 2013, (0124199542).
^{25.} Frederick M. Ausubel (ed.). Current Protocols in Molecular Biology (CPMB). John Wiley and Sons 2014, (9780471503385).
^{26.} John E. Coligan (ed.). Current Protocols in Protein Science (CPPS). John Wiley and Sons, Inc.; 2005,
^{27.} John E. Coligan ADA M Kruisbeek, David H Margulies, Ethan M Shevach, Warren Strobe, (eds.) Current Protocols in Immunology (CPI). John Wiley and Sons, Inc.; 2003, (9780471142737).
^{28.} McDonald R., et al. Pharmacokinetics of concentrated naloxone nasal spray for opioid overdose reversal: Phase I healthy volunteer study. Addiction. 2018; 113(3):484-493.
^{29.} Cohen P. A., et al. Pharmaceutical quantities of yohimbine found in dietary supplements in the USA. Drug Test Anal. 2016; 8(3-4):357-369.
^{30.} Anderson Colin, et al. Case Study: Two Fatal Case Reports of Acute Yohimbine Intoxication. Journal of Analytical Toxicology. 2013; 37(8):611-614.
^{31.} Drugs.com. Yohimbe. https://www.drugs.com/npp/yohimbe.html. Accessed Jun. 5, 2023.
^{32.} Morgan C. A., 3rd, et al. Yohimbine facilitated acoustic startle in combat veterans with post-traumatic stress disorder. Psychopharmacology (Berl). 1995; 117(4):466-471.
^{33.} Kaplan J. S., et al. Avoidant coping in panic disorder: a yohimbine biological challenge study. Anxiety Stress Coping. 2012; 25(4):425-442.
^{34.} Sommer M., et al. Psychological and neuroendocrine responses to social stress and to the administration of the alpha-2-receptor antagonist, yohimbine, in highly trained endurance athletes in comparison to untrained healthy controls. Pharmacopsychiatry. 2011; 44(4):129-134.
^{35.} Meyerbroeker K., et al. Does yohimbine hydrochloride facilitate fear extinction in virtual reality treatment of fear of flying? A randomized placebo-controlled trial. Psychother Psychosom. 2012; 81(1):29-37.
^{36.} Shen S. G., et al. Effects of alpha-adrenoreceptor antagonists on apoptosis and proliferation of pancreatic cancer cells in vitro. World J Gastroenterol. 2008; 14(15):2358-2363.
^{37.} DEA. Xylazine PDF. https://www.deadiversion.usdoj.gov/drug_chem_info/Xylazine.pdf. Accessed Jun. 5, 2023.
^{38.} Drugbank Online. Adrenergic alpha-2 Receptor Antagonists. https://go.drugbank.com/categories/DBCAT001356. Accessed Jun. 8, 2023.
^{39.} DEA. US Drug Enforcement Administration The Growing Threat of Xylazine and its Mixture with Illicit Drugs. https://www.dea.gov/sites/default/files/2022-12/The%20Growing%20Threat%200f%20Xylazine%20and%20its%20Mixture%20with%20lllicit%20Drugs.pdf.2024.
^{40.} CDC. What You Should Know About Xylazine. Centers for Disease Control. https://www.cdc.gov/overdose-prevention/about/what-you-should-know-about-xylazine.html.2024.
^{41.} Warp P. V., et al. A Confirmed Case of Xylazine-Induced Skin Ulcers in a Person Who Injects Drugs in Miami, Florida, USA. Res Sq. 2023.
^{42.} GREENE S. A., THURMON J. C. Xylazine—a review of its pharmacology and use in veterinary medicine. Journal of Veterinary Pharmacology and Therapeutics. 1988; 11(4):295-313.
^{43.} Nunez J., et al. Xylazine, a Veterinary Tranquilizer, Detected in 42 Accidental Fentanyl Intoxication Deaths. Am J Forensic Med Pathol. 2021; 42(1):9-11.
^{44.} Green T. C., et al. An assessment of the limits of detection, sensitivity and specificity of three devices for public health-based drug checking of fentanyl in street-acquired samples. Int J Drug Policy. 2020; 77:102661.
^{45.} Jansson T., et al. STANDING SEDATION WITH XYLAZINE AND REVERSAL WITH YOHIMBINE IN JUVENILE ASIAN ELEPHANTS (ELEPHAS MAXIMUS). J Zoo Wild/Med. 2021; 52(2):437-444.
^{46.} Jarosinski S. K., et al. The effects of xylazine or detomidine when used as a pre-anesthetic sedative on recovery quality and duration in horses undergoing elective equine castration. Can Vet J. 2021; 62(9):982-986.
^{47.} Jacobson E. R., et al. Effects of yohimbine on combined xylazine-ketamine-induced sedation and immobilization in juvenile African elephants. J Am Vet Med Assoc. 1985; 187(11):1195-1198.
^{48.} Curley D. E., et al. Yohimbine as a pharmacological probe for alcohol research: a systematic review of rodent and human studies. Neuropsychopharmacology. 2022; 47(12):2111-2122.
^{49.} Haass-Koffler C. L., et al. Mifepristone as a pharmacological intervention for stress-induced alcohol craving: A human laboratory study. Addict Biol. 2023; 28(7):e13288.
^{50.} Stanford S. C. Central noradrenergic neurones and stress. Pharmacol Ther. 1995; 68(2):297-242.
^{51.} Simms J. A., et al. Mifepristone in the central nucleus of the amygdala reduces yohimbine stress-induced reinstatement of ethanol-seeking. Neuropsychopharmacology. 2012; 37(4):906-918.
^{52.} Shepard J. D., et al. The anxiogenic drug yohimbine reinstates methamphetamine seeking in a rat model of drug relapse. Biol Psychiatry. 2004; 55(11):1082-1089.
^{53.} Marinelli P. W., et al. The CRF1 receptor antagonist antalarmin attenuates yohimbine-induced increases in operant alcohol self-administration and reinstatement of alcohol seeking in rats. Psychopharmacology (Berl). 2007; 195(3):345-355.
^{54.} Richards J. K., et al. Inhibition of orexin-1/hypocretin-1 receptors inhibits yohimbine-induced reinstatement of ethanol and sucrose seeking in Long-Evans rats. Psychopharmacology (Berl). 2008; 199(1):109-117.
^{55.} Cippitelli A., et al. Increase of brain endocannabinoid anandamide levels by FAAH inhibition and alcohol abuse behaviours in the rat. Psychopharmacology (Berl). 2008; 198(4):449-460.
^{56.} Cameron O. G., et al. Effects of yohimbine on cerebral blood flow, symptoms, and physiological functions in humans. Psychosom Med. 2000; 62(4):549-559.
^{57.} Woodcock E. A., et al. Pharmacological stress impairs working memory performance and attenuates dorsolateral prefrontal cortex glutamate modulation. Neuroimage. 2019; 186:437-445.
^{58.} Umhau J. C., et al. Pharmacologically induced alcohol craving in treatment seeking alcoholics correlates with alcoholism severity, but is insensitive to acamprosate. Neuropsychopharmacology. 2011; 36(6):1178-1186.
^{59.} Greenwald M. K., et al. Yohimbine increases opioid-seeking behavior in heroin-dependent, buprenorphine-maintained individuals. Psychopharmacology (Berl). 2013; 225(4):811-824.
^{60.} Woodcock E. A., et al. A neurobiological correlate of stress-induced nicotine-seeking behavior among cigarette smokers. Addict Biol. 2020; 25(4):e12819.
^{61.} GlobeNewswire. Vantage Market Research: Naloxone Market Size & Share to Surpass $ 743.25 Million by 2028|Vantage Market Research. https://www.globenewswire.com/en/news-release/2023/04/11/2644168/0/en/Naloxone-Market-Size-Share-to-Surpass-743-25-Million-by-2028-Vantage-Market-Research.html.2024.
^{62.} MayoClinic. Mayo Clinc: Alcohol Poisoning Signs and Symptoms. https://www.mayoclinic.org/diseases-conditions/alcohol-poisoning/symptoms-causes/syc-20354386.2024.
^{63.} Medscape. Benzodiazepine Toxicity. https://emedicine.medscape.com/article/813255-overview?form=fpf.2024.
^{64.} Orhurhu V. J., et al. Ketamine Toxicity. In: StatPearls. Treasure Island (FL): StatPearls Publishing Copyright © 2024, StatPearls Publishing LLC.; 2024.
^{65.} Lipman N. S., et al. Reversal of ketamine/xylazine anesthesia in the rabbit with yohimbine. Lab Anim Sci. 1987; 37(4):474-477.
^{66.} Hsu W. H., Lu Z. X. Effect of yohimbine on xylazine-ketamine anesthesia in cats. J Am Vet Med Assoc. 1984; 185(8):886-888.
^{67.} Hsu W. H., et al. Xylazine-ketamine-induced anesthesia in rats and its antagonism by yohimbine. J Am Vet Med Assoc. 1986; 189(9):1040-1043.
^{68.} Owen Quinn. ABC-News: Overdose deaths cost US $1 trillion annually, bipartisan report finds. https://abcnews.go.com/Politics/overdose-deaths-cost-us-trillion-annually-bipartisan-report/story?id=82726941.2024.
^{69.} Langabeer J. R., et al. County-level factors underlying opioid mortality in the United States. Subst Abus. 2022; 43(1):76-82.
^{70.} Doherty T. J., et al. Cardiopulmonary effects of xylazine and yohimbine in laterally recumbent sheep. Can J Vet Res. 1986; 50(4):517-521.
^{71.} Garcia-Villar R., et al. The pharmacokinetics of xylazine hydrochloride: an interspecific study. J Vet Pharmacol Ther. 1981; 4(2):87-92.
^{72.} Hedner T., et al. Yohimbine pharmacokinetics and interaction with the sympathetic nervous system in normal volunteers. Eur J Clin Pharmacol. 1992; 43(6):651-656.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize or be able to ascertain using no more than routine experimentation, many equivalents to the specific embodiments described herein. Such equivalents are intended to be encompassed by the following exemplary claims.

We claim:

1. A method for preventing and/or treating a subject having a substance overdose or suspected of having a substance overdose, the method comprising administering an alpha-2 (α₂) adrenergic antagonist to the subject, whereby one or more signs or symptoms of the substance overdose are prevented or reduced.

2. The method of claim 1, wherein said α₂ adrenergic antagonist is selected from the group consisting of: a blocker, an orthosteric antagonist, allosteric antagonist, conformation changing binder, a modulator, a partial inverse agonist/inverse agonist, or a combination thereof.

3. The method of claim 1, wherein said α₂ adrenergic antagonist comprises yohimbine or a pharmaceutically acceptable salt and/or hydrate or solvate thereof.

4. The method of claim 1, wherein the substance overdose comprises one or more unknown drugs or substances in the subject.

5. The method of claim 1, wherein the administration of the α₂ adrenergic antagonist results in a partial or complete reversal of symptoms of the substance overdose at least contributed to by one or more drugs included in said substance comprising an opioid or a synthetic opioid, xylazine and xylazine congeners, an alcohol, benzodiazepines, or a combination thereof.

6. The method of claim 1, wherein the substance comprises fentanyl, heroin, hydromorphone, methadone, buprenorphine; morphine, codeine, xylazine and xylazine congeners, or a combination thereof.

7. The method of claim 1, wherein the one or more signs or symptoms of the substance overdose are selected from the group consisting of: dry mouth, drowsiness, hypertension and/or hypotension, tachycardia, bradycardia, hyperglycemia, reduced heart rate, hypothermia, coma, respiratory depression, and/or dysrhythmia.

8. The method of claim 1, wherein said α₂ adrenergic antagonist is administered in a fast-acting formulation capable of providing antagonist plasma concentration levels≥50% of peak concentrations within 60 minutes of the administration.

9. The method of claim 8, wherein said formulation is provided in a form selected from the group consisting of: a buccal film, oral tablet, sublingual tablet, nasal spray, inhaled aerosol, inhaled solid powder, intravenous injectable, intra-arterial injectable, intracardiac injectable, hydrogel or organogel, powder, intramuscular injectable, subcutaneous injectable, intradermal injectable, eye (ocular) formulation drops, ocular mist, or fast absorbing topical formulation.

10. The method of claim 1, wherein the α₂ adrenergic antagonist is co-administered with Naloxone.

11. The method of claim 1, wherein the α₂ adrenergic antagonist is administered after an initial administration of Naloxone to the subject was ineffective in reversing and/or in diminishing the symptoms of substance abuse.

12. The method of claim 1, wherein the α₂ adrenergic antagonist is administered as a prophylactic in a subject exhibiting one or more signs or symptoms of chronic substance abuse selected from the group consisting of: soft tissue injuries, skin ulcers, abscesses, and/or tissue necrosis.

13. A lifesaving kit for preventing and/or for treating a subject having a substance overdose or suspected of having a substance overdose, the kit comprising:
(1) an alpha-2 (α₂) adrenergic antagonist in a fast-acting pharmaceutical formulation suitable for an emergency administration to the subject; and
(2) instructions for use.

14. The lifesaving kit of claim 13, further comprising an aerosol dispenser device, a particulate dispenser device, an autoinjector device, a sublingual tablet, an eyedropper device, or a combination thereof.

15. The lifesaving kit of claim 13, wherein the α₂ adrenergic antagonist comprises a blocker, an orthosteric antagonist, allosteric antagonist, conformation changing binder, a modulator, a partial inverse agonist/inverse agonist, or a combination thereof.

16. The lifesaving kit of claim 13, wherein the α₂ adrenergic antagonist comprises yohimbine or a pharmaceutically acceptable salt and/or hydrate or solvate thereof.

17. A system for preventing a prevalence or a rise in a number of occurrences of overdoses in a population of subjects, each of the subjects in the population spread over a geographic area, the system comprising:
(1) a plurality of kits including an alpha-2 (α₂) adrenergic antagonist in a fast-acting pharmaceutical formulation suitable for an emergency administration to the subject suffering from an overdose or suspected of suffering from an overdose;
(2) a deployment of the plurality of kits over the geographical area with a greater number or a greater geographical concentration of each of the kits from the plurality deployed in one or more geographical areas known to have a higher incidence of a drug overdose.

18. The system of claim 17, wherein the system is further comprising: (3) determining the one or more geographical areas known to have a higher incidence of a drug overdose based upon an emergency ambulance and/or hospital emergency room record for each of the one or more geographical areas; and (4) repeating the deployment of (2) to provide a sharper focus of more of the kits or a greater concentration of the kits in the geographical areas known to have a higher incidence of a drug overdose.

19. The system of claim 17, further comprising (5) measuring the incidence of a drug overdose based upon an emergency record for each of the one or more geographical areas and determining if an incidence of one or more fatal drug overdoses decreases after the deployment in (2).
